(19) United States
(12) Patent Application Publication
(10) Pub. No.: US 2007/0254307 A1
(43) Pub. Date: Nov. 1, 2007

(54) Method for Estimation of Location of Active Sites of Biopolymers Based on Virtual Library Screening

(75) Inventor: David Kita, Milpitas, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Verseon, Fremont, CA (US)

(21) Appl. No.: 11/739,578

(22) Filed: Apr. 24, 2007

Related U.S. Application Data
(60) Provisional application No. 60/795,678, filed on Apr. 28, 2006.

(51) Int. Cl. G06F 19/00; G01N 33/53

(52) U.S. Cl. 435/7.1; 702/19

(57) ABSTRACT

A method and apparatus for estimating a location of one or more active sites on a target biopolymer molecular subset. The collective results for the likelihood of molecular combination between a collection of molecular subsets and the target biopolymer molecular subset are analyzed. The computational method utilizes an electrostatic affinity score for different configurations between the target biopolymer molecular subset and the molecular subsets of the collection. Favorable configurations are determined based on the affinity scores. These favorable configurations are then used to determine interaction loci, which are associated with regions of the target biopolymer molecular subset having a high likelihood of molecular combination. The locations of the active sites are then estimated from the interaction loci.

### CROSS-REFERENCES TO RELATED APPLICATIONS

The present application claims priority from and is a nonprovisional application of U.S. Provisional Application No. 60/795,678 (Attorney Docket No. 021986-001500US), entitled “Method for Estimation of Location of Active Sites of Biopolymers Based on Virtual Library Screening” filed Apr. 28, 2006, the entire contents of which are herein incorporated by reference for all purposes.

A related patent is U.S. Pat. No. 6,970,790 entitled “Method and Apparatus for Analysis of Molecular Combination Based on Computational Estimation of the Electrostatic Affinity Using a Basis Expansion”, to Kita et al. (hereinafter “Kita I”), the entire contents of which is herein incorporated by reference for all purposes.

Another related application, incorporated by reference for all purposes, is U.S. patent application Ser. No. 10/966,160 filed Oct. 14, 2004 (Attorney Docket No. 021986-000610US) entitled “Method and Apparatus for Analysis of Molecular Combination Based on Computations of Shape Complementarity Using Basis Expansions” to Kita et al. (hereinafter “Kita II”).

### BACKGROUND OF THE INVENTION

The present invention generally relates to bioinformatics, proteomics, molecular modeling, computer aided molecular design (CAMD), and more specifically computer aided drug design (CADD) and computational modeling of molecular combinations in the context of active site identification.

Identifying the location of one or more active sites on a protein (i.e., ligand-binding or protein-binding domains) is of fundamental importance for a range of drug discovery applications including molecular docking, de novo drug design and structural identification and comparison of functional sites across proteins. Among the many uses of such identification is to reduce the cost, time and effort of drug development.

A conventional drug discovery process has many limitations. Discovering a new drug to treat or cure some biological condition, is a lengthy and expensive process, typically taking on average 12 years and $800 million per drug, and taking possibly up to 15 years or more and $1 billion to complete in some cases.

A goal of a drug discovery process is to identify and characterize a chemical compound or ligand biomolecule that affects the function of one or more other biomolecules (i.e., a drug “target”) in an organism, usually a biopolymer, via a potential molecular interaction or combination. Herein the term biopolymer refers to a macromolecule that comprises one or more of a protein, nucleic acid (DNA or RNA), peptide or nucleotide sequence or any portions or fragments thereof. Herein the term biomolecule refers to a chemical entity that comprises one or more of a biopolymer, carbohydrate, hormone, or other molecule or chemical compound, either inorganic or organic, including, but not limited to, synthetic, medicinal, drug-like, or natural compounds, or any portions or fragments thereof.

The target molecule is typically a disease-related target protein or nucleic acid for which it is desired to affect a change in function, structure, and/or chemical activity in order to aid in the treatment of a patient disease or other disorder. In other cases, the target is a biomolecule found in a disease-causing organism, such as a virus, bacteria, or parasite, that when affected by the drug will affect the survival or activity of the infectious organism. In yet other cases, the target is a biomolecule of a defective or harmful cell such as a cancer cell. In yet other cases the target is an antigen or other environmental chemical agent that may induce an allergic reaction or other undesired immunological or biological response.

The ligand is typically a small molecule drug or chemical compound with desired drug-like properties in terms of potency, low toxicity, membrane permeability, solubility, chemical/metabolic stability, etc. In other cases, the ligand may be biologic such as an injected protein-based or peptide-based drug or even another full-fledged protein. In yet other cases the ligand may be a chemical substrate of a target enzyme. The ligand may even be covalently bound to the target or may in fact be a portion of the protein, e.g., protein secondary structure component, protein domain containing or near an active site, protein sub-unit of an appropriate protein quaternary structure, etc.

Throughout the remainder of the background discussion, unless otherwise specifically differentiated, a (potential) molecular combination will feature one ligand and one target, the ligand and target will be separate chemical entities, and the ligand will be assumed to be a chemical compound while the target will be a biological protein (mutant or wild type). Note that the frequency of nucleic acids (both DNA/RNA) as targets will likely increase in coming years as advances in gene therapy and pathogenic microbiology progress. Also the term “molecular complex” will refer to the bound state between the target and ligand when interacting with one another in the midst of a suitable (often aqueous) environment. A “potential” molecular complex refers to a bound state that may occur albeit with low probability and therefore may or may not actually form under normal conditions.

The drug discovery process itself typically includes four different sub-processes: (1) target validation; (2) lead generation/optimization; (3) pre-clinical testing; and (4) clinical trials and approval.

Target validation includes determination of one or more targets that have disease relevance and usually takes two-and-a-half years to complete. Results of the target validation phase might include a determination that the presence or action of the target molecule in an organism causes or influences some effect that initiates, exacerbates, or contributes to a disease for which a cure or treatment is sought. In some cases a natural binder or substrate for the target may also be determined via experimental methods. It may also be desired in the target validation phase to identify or characterize one or more active sites on the biopolymer.

A description of the concepts of active sites follows. Herein, “active site” refers to a volumetric region enclosing a plurality of residues, or other structural components of a biopolymer, where such residues may form strong interactions or contacts with one or more molecular substrates or binders at thermodynamic equilibrium under standard environmental conditions. In many cases, the substrates in question are themselves other biopolymers. Sometimes the active site is also referred to as the binding site or the binding pocket. While a biopolymer may have one or more such active sites, often each active site corresponds to some (but not always distinct) function(s) of the target biopolymer. In the case of target proteins one or more active sites may be highly conserved in terms of both internal structure and location on the protein surface across other family members. It is this functional correspondence as well as potential conservation across homologous proteins that makes active site characterization an important part of target validation.

Note that an active site of a biopolymer may be highly flexible and may adopt very different conformations based on which substrates are interacting with the target biopolymer. In some cases, it may be possible to identify active sites through chemo-informatic or experimental means. In other cases this is not easily accomplished and a virtual method using computational analysis of the biopolymer structure is desired.

FIGS. 1A and 1B illustrate a protein having active sites. In FIG. 1A, a protein 110 is shown. In this example, the protein is human dihydrofolate reductase (DHFR), an enzyme involved in the DNA synthesis pathway. A surface 120 is a solvent accessible surface made by rolling a small water probe across the surface atoms of the protein exposed to solvent. The shaded sub-region 140 is in fact the active site of the catalytic domain of DHFR. In FIG. 1B, the same information is displayed but now in addition a molecule 160 is shown bound to protein 110 in the active site 140. Molecule 160 is methotrexate, a known binder to DHFR. The structural pose of molecule 160 of this example is a snapshot of its conformation from an X-ray crystal structure (PDB entry: 4dfr).

Returning to a description of the drug discovery process, lead generation typically involves the identification of lead compounds that can bind to the target molecule and thereby alter the effects of the target through either activation, deactivation, catalysis, or inhibition of the function of the target, in which case the lead would be a viewed as a suitable candidate ligand to be used in the drug application process. Lead optimization involves the chemical and structural refinement of lead candidates into drug precursors in order to improve binding affinity to the desired target, increase selectivity, and also to address issues of toxicity, solubility, and metabolism. Together lead generation and lead optimization typically takes about three years to complete and might result in one or more chemically distinct leads for further consideration.

In pre-clinical testing, biochemical assays and animal models are used to test the selected leads for various pharmacokinetic factors related to drug absorption, distribution, metabolism, excretion, toxicity, side effects, and required dosages. This pre-clinical testing takes approximately one year. After the pre-clinical testing period, clinical trials and approval take another six to eight or more years during which the drug candidates are tested on human subjects for safety and efficacy.

Rational drug design generally uses structural information about drug targets (structure-based) and/or their natural ligands (ligand-based) as a basis for the design of effective lead candidate generation and optimization. Structure-based rational drug design generally utilizes a three-dimensional model of the structure for the target. For target proteins or nucleic acids such structures may be as the result of X-ray crystallography/NMR or other measurement procedures or may result from homology modeling, analysis of protein motifs and conserved domains, and/or computational modeling of protein folding or the nucleic acid equivalent. Model-built structures are often all that is available when considering many membrane-associated target proteins, e.g., GPCRs and ion-channels. The structure of a ligand may be generated in a similar manner or may instead be constructed ab initio from a known 2-D chemical representation using fundamental physics and chemistry principles, provided the ligand is not a biopolymer. As it relates to the present invention, such structural information can be very useful when attempting to predict the locations of one or more biopolymer active sites.

Rational drug design may incorporate the use of any of a number of computational components ranging from computational modeling of target-ligand molecular interactions and combinations to lead optimization to computational prediction of desired drug-like biological properties. Indeed, the computational identification of biopolymer (protein) active site(s) would also fit into the purview of rational drug design. The use of computational modeling in the context of rational drug design has been largely motivated by a desire to both reduce the required time and to improve the focus and efficiency of drug research and development, by avoiding often time consuming and costly efforts in biological “wet” lab testing and the like.

Computational modeling of target-ligand molecular combinations in the context of lead generation may also involve the large-scale in-silico screening of compound libraries (i.e., library screening), whether the libraries are virtually generated and stored as one or more compound structural databases or constructed via combinatorial chemistry and organic synthesis, using computational methods to rank a selected subset of ligands based on computational prediction of bioactivity (or an equivalent measure) with respect to the intended target molecule.

Throughout the text, the term “binding mode” refers to the 3-D molecular structure of a potential molecular complex in a bound state at or near a minimum of the binding energy (i.e., maximum of the binding affinity), where the term “binding energy” (sometimes interchanged with “binding affinity” or “binding free energy”) refers to the change in free energy of a molecular system upon formation of a potential molecular complex, i.e., the transition from an unbound to a (potential) bound state for the ligand and target. Here the term free energy generally refers to both enthalpic and entropic effects as the result of physical interactions between the constituent atoms and bonds of the molecules between themselves (i.e., both inter-molecular and intra-molecular interactions) and with their surrounding environment. Examples of the free energy are the Gibbs free energy encountered in the canonical or grand canonical ensembles of equilibrium statistical mechanics.

In general, the optimal binding free energy of a given target-ligand pair directly correlates to the likelihood of formation of a potential molecular complex between the two molecules in chemical equilibrium, though, in truth, the binding free energy describes an ensemble of (putative) complexed structures and not one single binding mode. However, in computational modeling it is usually assumed that the change in free energy is dominated by a single structure corresponding to a minimal energy. This is certainly true for tight binders (pK ˜0.1 to 10 nanomolar) but questionable for weak ones (pK ˜10-100 micromolar). The dominating structure is usually taken to be the binding mode. In some cases it may be necessary to consider more than one alternative-binding mode when the associated system states are nearly degenerate in terms of energy.

It is desirable in the drug discovery process to identify quickly and efficiently the optimal docking configurations, i.e., binding modes, of two molecules or parts of molecules. Efficiency is especially relevant in the lead generation and lead optimization stages for a drug discovery pipeline, where it is desirable to accurately predict the binding mode for possibly millions of potential molecular complexes, before submitting promising candidates to further analysis.

Binding modes and binding affinity are of direct interest to drug discovery and rational drug design because they often help indicate how well a potential drug candidate may serve its purpose. Furthermore, where the binding mode is determinable, the action of the drug on the target can be better understood. Such understanding may be useful when, for example, it is desirable to further modify one or more characteristics of the ligand so as to improve its potency (with respect to the target), binding specificity (with respect to other targets), or other chemical and metabolic properties.

A number of laboratory methods exist for measuring or estimating affinity between a target molecule and a ligand. Often the target might be first isolated and then mixed with the ligand in vitro and the molecular interaction assessed experimentally such as in the myriad biochemical and functional assays associated with high throughput screening. However, such methods are most useful where the target is simple to isolate, the ligand is simple to manufacture and the molecular interaction easily measured, but is more problematic when the target cannot be easily isolated, isolation interferes with the biological process or disease pathway, the ligand is difficult to synthesize in sufficient quantity, or where the particular target or ligand is not well characterized ahead of time. In the latter case, many thousands or millions of experiments might be needed for all possible combinations of the target and ligands, making the use of laboratory methods unfeasible.

While a number of attempts have been made to resolve this bottleneck by first using specialized knowledge of various chemical and biological properties of the target (or even related targets such as protein family members) and/or one or more already known natural binders or substrates to the target, to reduce the number of combinations required for lab processing, this is still impractical and too expensive in most cases. Instead of actually combining molecules in a laboratory setting and measuring experimental results, another approach is to use computers to simulate or characterize molecular interactions between two or more molecules (i.e., molecular combinations modeled in silico). The use of computational methods to assess molecular combinations and interactions is usually associated with one or more stages of rational drug design, whether structure-based, ligand-based, or both.

The computational prediction of one or more binding modes and/or the computational assessment of the nature of a molecular combination and the likelihood of formation of a potential molecular complex is generally associated with the term “docking” in the art. To date, conventional “docking” methods have included a wide variety of computational techniques as described below under the heading “REFERENCES AND PRIOR ART”.

Whatever the choice of computational docking method there are inherent trade-offs between the computational complexity of both the underlying molecular models and the intrinsic numerical algorithms, and the amount of compute resources (time, number of CPUs, number of simulations) that must be allocated to process each molecular combination. For example, while highly sophisticated molecular dynamics simulations (MD) of the two molecules surrounded by explicit water molecules and evolved over trillions of time steps may lead to higher accuracy in modeling the potential molecular combination, the resultant computational cost (i.e., time and compute power) is so enormous that such simulations are intractable for use with more than just a few molecular combinations.

One major distinction amongst docking methods as applied to computational modeling of molecular combinations is whether the ligand and target structures remain rigid throughout the course of the simulation (i.e., rigid-body docking) vs. the ligand and/or target being allowed to change their molecular conformations (i.e., flexible docking). In general, the latter scenario involves more computational complexity, though flexible docking may often achieve higher accuracy than rigid-body docking when modeling various molecular combinations.

That being said rigid-body docking can provide valuable insight into the nature of a molecular combination and/or the likelihood of formation of a potential molecular complex and has many potential uses within the context of rational drug discovery. For instance rigid-body docking may be appropriate for docking small, rigid molecules (or molecular fragments) to a simple protein with a well-defined, nearly rigid active site. As another example, rigid-body docking may also be used to more efficiently and rapidly screen out a subset of likely non-active ligands in a molecule library for a given target, and then applying more onerous flexible docking procedures to the surviving candidate molecules. Rigid-body docking may also be suitable for de novo ligand design and combinatorial library design.

Moreover, in order to better predict the binding mode and better assess the nature and/or likelihood of a molecular combination when one or both molecules are likely to be flexible, rigid-body docking can be used in conjunction with a process for generating likely yet distinct molecular conformers of one or both molecules for straightforward and efficient virtual screening of a molecule library against a target molecule. However, as will be discussed, even rigid body docking of molecular combinations can be computationally expensive and thus there is a clear need for better and more efficient computational methods based on rigid body docking when assessing the nature and/or likelihood of molecular combinations.

As outlined in the section entitled “REFERENCES AND PRIOR ART”, conventional computational methods for predicting binding modes and assessing the nature and/or likelihood of molecular combinations in the context of rigid-body docking include a wide variety of techniques. These include methods based on pattern matching (often graph-based), maximization of shape complementarity (i.e., shape correlations), geometric hashing, pose clustering, and even the use of one or more flexible docking methods with the simplifying run-time condition that both molecules are rigid.

One interesting class of rigid-body docking techniques are those based on the maximization of shape complementarity via evaluation of the spatial correlation between two representative molecular surfaces at different relative positions and orientations. Here the term “shape complementarity” measures the geometric fit or correlation between the molecular shapes of two molecules. The concept can be generalized to any two objects. For example, two pieces of a jigsaw puzzle that fit each other exhibit strong shape complementarity.

Shape complementarity based methods, while typically treating molecules as rigid and thus perhaps less rigorous than their flexible docking counterparts, especially in the context of flexible molecules, is still potentially valuable for the fast, efficient screening of two molecules in order to make a preliminary assessment of the nature and/or likelihood of formation of a potential molecular complex of the two molecules or to make an initial prediction of the preferred binding mode for the molecular combination. Such a preliminary assessment may significantly reduce the number of candidates that must be further screened in silico by another more computationally costly docking method.

A full description of various existing methods involving shape complementarity can be found in the background sections of Kita I and Kita II. The invention disclosed in Kita I additionally includes electrostatic interactions when assessing whether or not a binding mode is favorable. Further background of the importance of electrostatic interactions in biology and chemistry can be found in, for example, the review article by Honig et al [35]. As mentioned in Kita I a combined analysis of shape complementarity and electrostatic affinity can result in a very powerful and efficient tool for modeling molecular combinations via rigid body transformations.

In summary, it is desirable in the drug discovery process to identify quickly and efficiently the optimal configurations, i.e., binding modes, of two molecules or parts of molecules. Efficiency is especially relevant in the lead generation and lead optimization stages for a drug discovery pipeline, where it may be desirable to accurately predict the binding mode and binding affinity for possibly millions of potential target-ligand molecular combinations, before submitting promising candidates to further analysis. As an adjoint to this, it is also desirable in the target validation phase to identify one or more active sites of a target biopolymer. In fact, there is a clear need to have more efficient systems and methods for computational prediction and characterization of biopolymer active sites with reasonable accuracy.

### REFERENCES AND PRIOR ART

Prior art in the field of the current invention is heavily documented: the following tries to summarize it.

Drews [1] provides a good overview of the current state of drug discovery. In [2] Abagyan and Totrov show the state of high throughput docking and scoring and its applications. Lamb et al [3] further teach a general approach to the design, docking, and virtual screening of multiple combinatorial libraries against a family of proteins, finally Waskowycz et al [4] describe the use of multiple computers to accelerate virtual screening of a large ligand library against a specific target by assigning groups of ligands to specific computers.
[1] J. Drews, “Drug Discovery: A Historical perspective,” Science 287, 1960-1964 (2000).
[2] Ruben Abagyan and Maxim Totrov, “High-throughput docking for lead generation”, Current Opinion in Chemical Biology 2001, 5:375-382.
[3] Lamb, M. L.; Burdick, K. W.; Toba, S.; Young, M. M.; Skillman, A. G. et al., “Design, docking, and evaluation of multiple libraries against multiple targets”, Proteins 2001, 42, 296-318.
[4] Waszkowycz, B., Perkins, T. D. J., Sykes, R. A., Li, J., “Large-scale virtual screening for discovering leads in the post-genomic era”, IBM Systems Journal, Vol. 40, No. 2 (2001).

There are a number of examples of software tools currently used to perform docking simulations. These methods involve a wide range of computational techniques, including use of a) rigid-body pattern-matching algorithms, either based on surface correlations, use of geometric hashing, pose clustering, or graph pattern-matching; b) fragmental-based methods, including incremental construction or “place and join” operators; c) stochastic optimization methods including use of Monte Carlo, simulated annealing, or genetic (or memetic) algorithms; d) molecular dynamics simulations or e) hybrids strategies derived thereof.

The earliest docking software tool was a graph-based rigid-body pattern-matching algorithm called DOCK [5][6][7] developed at UCSF back in 1982 (v1.0) and now up to v5.0 (with extensions to include incremental construction). Other examples of graph-based pattern-matching algorithms include CLIX [8] (which in turn uses GRID [9]), FLOG [10] and LIGIN [11].
[5] Shoichet, B. K., Bodian, D. L. and Kuntz, I. D., “Molecular docking using shape descriptors”, J. Comp. Chem., Vol. 13 No. 3, 380-397 (1992).
[6] Meng, E. C., Gschwend, D. A., Blaney, J. M., and I. D. Kuntz, “Orientational sampling and rigid-body minimization in molecular docking”, Proteins: Structure, Function, and Genetics, Vol. 17, 266-278 (1993).
[7] Ewing, T. J. A. and Kuntz, I. D., “Critical Evaluation of Search Algorithms for Automated Molecular Docking and Database Screening”, J. Computational Chemistry, Vol. 18 No. 9, 1175-1189 (1997).
[8] Lawrence, M. C. and Davis, P. C.; “CLIX: A Search Algorithm for Finding Novel Ligands Capable of Binding Proteins of Known Three-Dimensional Structure”, Proteins, Vol. 12, 31-41 (1992).
[9] Kastenholz, M. A., Pastor, M., Cruciani, G., Haaksma, E. E. J., Fox, T., “GRID/CPCA: A new computational tool to design selective ligands”, J. Medicinal Chemistry, Vol. 43, 3033-3044 (2000).
[10] Miller, M. D., Kearsley, S. K., Underwood, D. J. and Sheridan, R. P., “FLOG: A System to Select ‘Quasi-Flexible’ Ligands Complementary to a Receptor of Known Three-Dimensional Structure”, J. Computer-Aided Molecular Design, Vol. 8 No. 2, 153-174 (1994).
[11] Sobolev, V., Wade, R. C., Vriend, G. and Edelman, M., “Molecular docking using surface complementarity”, Proteins, Vol. 25, 120-129 (1996)

Other rigid-body pattern-matching docking software tools include the shape-based correlation methods of FTDOCK [12] and HEX [13], the geometric hashing of Fischer et al. [14], or the pose clustering of Rarey et al [15].
[12] Katchalski-Katzir, E., Shariv, I., Eisenstein, M., Friesem, A. A., Aflalo, C., and Vakser, I. A., “Molecular surface recognition: Determination of geometric fit between proteins and their ligands by correlation techniques”, Proceedings of the National Academy of Sciences of the United States of America, Vol. 89 No. 6, 2195-2199 (1992).
[13] Ritchie, D. W. and Kemp, G. J. L., “Fast Computation, Rotation, and Comparison of Low Resolution Spherical Harmonic Molecular Surfaces”, J. Computational Chemistry, Vol. 20 No. 4, 383-395 (1999).
[14] Fischer, D., Norel, R., Wolfson, H. and Nussinov, R., “Surface motifs by a computer vision technique: searches, detection, and implications for protein-ligand recognition”, Proteins, Vol. 16, 278-292 (1993).
[15] Rarey, M., Wefing, S., and Lengauer, T., “Placement of medium-sized molecular fragments into active sites of proteins”, J. Computer-Aided Molecular Design, Vol. 10, 41-54 (1996).

In general, rigid-body pattern-matching algorithms assume that both the target and ligand are rigid (i.e., not flexible) and hence may be appropriate for docking small, rigid molecules (or molecular fragments) to a simple protein with a well-defined, nearly rigid active site. Thus this class of docking tools may be suitable for de novo ligand design, combinatorial library design, or straightforward rigid-body screening of a molecule library containing multiple conformers per ligand.

Incremental construction based docking software tools include FlexX [16][17] from Tripos (licensed from EMBL), Hammerhead [18], DOCK v4.0 [7] (as an option), and the non-greedy, backtracking algorithm of Leach et al. [19]. Programs using incremental construction in the context of de novo ligand design include LUDI [20] (from Accelrys) and GrowMol [21]. Docking software tools based on “place and join” strategies include DesJarlais et al. [22].
[16] Kramer, B., Rarey, M. and Lengauer, T., “Evaluation of the FlexX incremental construction algorithm for protein-ligand docking”, Proteins, Vol. 37, 228-241 (1999).
[17] Rarey, M., Kramer, B., Lengauer, T., and Klebe, G., “A Fast Flexible Docking Method Using An Incremental Construction Algorithm”, J. Mol. Biol., Vol. 261, 470-489 (1996).
[18] Welch, W., Ruppert, J. and Jain, A. N., “Hammerhead: Fast, fully automated docking of flexible ligands to protein binding sites”, Chemical Biology, Vol. 3, 449-462 (1996).
[19] Leach, A. R., Kuntz, I. D., “Conformational Analysis of Flexible Ligands in Macromolecular Receptor Sites”, J. Comp. Chem., Vol. 13, 730-748 (1992).
[20] Bohm, H. J., “The computer program LUDI: a new method for the de novo design of enzyme inhibitors”, J. Computer-Aided Molecular Design, Vol. 6, 61-78 (1992).
[21] Bohacek, R. S, and McMartin, C., “Multiple Highly Diverse Structures Complementary to Enzyme Binding Sites: Results of Extensive Application of a de Novo Design Method Incorporating Combinatorial Growth”, J. American Chemical Society, Vol. 116, 5560-5571 (1994).
[22] DesJarlais, R. L., Sheridan, R. P., Dixon, J. S., Kuntz, I. D., and Venkataraghavan, R., “Docking Flexible Ligands to Macromolecular Receptors by Molecular Shape”, J. Med. Chem., Vol. 29, 2149-2153 (1986).

Incremental construction algorithms may be used to model docking of flexible ligands to a rigid target molecule with a well-characterized active site. They may be used when screening a library of flexible ligands against one or more targets. They are often comparatively less compute intensive, yet consequently less accurate, than many of their stochastic optimization based competitors. However, even FlexX may take on order of <1-2 minutes to process one target-ligand combination and thus may still be computationally onerous depending on the size of the library (e.g., tens of millions or more compounds). Recently FlexX was extended to FlexE [23] to attempt to account for partial flexibility of the target molecule's active site via use of user-defined ensembles of certain active site rotamers.
[23] Claussen, H., Buning, C., Rarey, M., and Lengauer, T., “FlexE: Efficient Molecular Docking Considering Protein Structure Variations”, J. Molecular Biology, Vol. 308, 377-395 (2001).

Computational docking software tools based on stochastic optimization include ICM [24] (from MolSoft), GLIDE [25] (from Schrodinger), and LigandFit [26] (from Accelrys), all based on modified Monte Carlo techniques, and AutoDock v.2.5 [27] (from Scripps Institute) based on simulated annealing. Others based on genetic or memetic algorithms include GOLD [28][29], DARWIN [30], and AutoDock v.3.0 [31] (also from Scripps).
[24] Abagyan, R. A., Totrov, M. M., and Kuznetsov, D. N., “Biased probability Monte Carlo conformational searches and electrostatic calculations for peptides and proteins”, J. Comp. Chem., Vol. 15, 488-506 (1994).
[25] Halgren, T. A., Murphy, R. B., Friesner, R. A., Beard, H. S., Frye, L. L., Pollard, W. T., and Banks, J. L., “Glide: a new approach for rapid, accurate docking and scoring. 2. Enrichment factors in database screening”, J Med. Chem., Vol. 47 No. 7, 1750-1759, (2004).
[26] Luty, B. A., Wasserman, Z. R., Stouten, P. F. W., Hodge, C. N., Zacharias, M., and McCammon, J. A., “Molecular Mechanics/Grid Method for the Evaluation of Ligand-Receptor Interactions”, J. Comp. Chem., Vol. 16, 454-464 (1995).
[27] Goodsell, D. S. and Olson, A. J., “Automated Docking of Substrates to Proteins by Simulated Annealing”, Proteins: Structure, Function, and Genetics, Vol. 8, 195-202 (1990).
[28] Jones, G., Willett, P. and Glen, R. C., “Molecular Recognition of Receptor Sites using a Genetic Algorithm with a Description of Desolvation”, J. Mol. Biol., Vol. 245, 43-53 (1995).
[29] Jones, G., Willett, P., Glen, R. C., Leach, A., and Taylor, R., “Development and Validation of a Genetic Algorithm for Flexible Docking”, J. Mol. Biol., Vol. 267, 727-748 (1997).
[30] Taylor, J. S, and Burnett, R. M., Proteins, Vol. 41, 173-191 (2000).
[31] Morris, G. M., Goodsell, D. S., Halliday, R. S., Huey, R., Hart, W. E., Belew, R. K. and Olson, A. J., “Automated Docking Using a Lamarckian Genetic Algorithm and an Empirical Binding Free Energy Function”, J. Comp. Chem., Vol. 19, 1639-1662 (1998).

Stochastic optimization-based methods may be used to model docking of flexible ligands to a target molecule. They generally use a molecular-mechanics based formulation of the affinity function and employ various strategies to search for one or more favorable system energy minima. They are often more compute intensive, yet also more robust, than their incremental construction competitors. As they are stochastic in nature, different runs or simulations may often result in different predictions. Traditionally most docking software tools using stochastic optimization assume the target to be nearly rigid (i.e., hydrogen bond donor and acceptor groups in the active site may rotate), since otherwise the combinatorial complexity increases rapidly making the problem difficult to robustly solve in reasonable time.

Molecular dynamics simulations have also been used in the context of computational modeling of target-ligand combinations. A molecular dynamics simulation refers to a simulation method devoted to the calculation of the time dependent behavior of a molecular system in order to investigate the structure, dynamics and thermodynamics of molecular systems. Examples include the implementations presented in Di Nola et al. [32], Mangoni et al. [33] and Luty et al. [26] (along with Monte Carlo). In principle, molecular dynamics simulations may be able to model protein flexibility to an arbitrary degree. On the other hand, they may also require evaluation of many fine-grained, time steps and are thus often very time-consuming (one order of hours or even days per target-ligand combination). They also often require user-interaction for selection of valid trajectories. Use of molecular dynamics simulations in lead discovery is therefore more suited to local minimization of predicted complexes featuring a small number of promising lead candidates.
[32] Di Nola, A., Berendsen, H. J. C., and Roccatano, D., “Molecular Dynamics Simulation of the Docking of Substrates to Proteins”, Proteins, Vol. 19, 174-182 (1994).
[33] Mangoni, M., Raccatano, D., and Di Nola, A., “Docking of Flexible Ligands to Flexible Receptors in Solution by Molecular Dynamics Simulation”, Proteins: Structure, Function, and Genetics, 35, 153-162, 1999;

Hybrid methods may involve use of rigid-body pattern matching techniques for fast screening of selected low-energy ligand conformations, followed by Monte Carlo torsional optimization of surviving poses, and finally even molecular dynamics refinement of a few choice ligand structures in combination with a (potentially) flexible protein active site. An example of this type of docking software strategy is Wang et al. [34].
[34] Wang, J., Kollman, P. A. and Kuntz, I. D., “Flexible ligand docking: A multi-step strategy approach”, Proteins, Vol. 36, 1-19 (1999).

A review discussing the importance of electrostatic interactions in biology and chemistry can be found in Honig et al. [35]. When modeling electrostatics interactions between molecules in an environment, especially in the context of molecular dynamics simulations or other molecular mechanics-based methods, the assignment of charges (full or partial) to various molecular components must be addressed. An example of a commonly used, and classically derived, method for assignment of partial charges is PARSE described in Sitkoff et al. [36]. An example of commonly used quantum mechanical based software packages for the purpose of the assignment of partial charges is MOPAC [37] and GAMESS [38].
[35] Honig, B., and Nicholls, A., “Classical Electrostatics in Biology and Chemistry”, Science, Vol. 268, 1144-1148 (1995).
[36] Sitkoff, D., Sharp, K. A., and Honig, B., in “Accurate Calculation of Hydration Free Energies Using Macroscopic Solvent Models”, J. Phys. Chem., Vol. 98, 1978-1988 (1994).
[37] J. J. P. Stewart, “MOPAC: A General Molecular Orbital Package” in Quantum Chemistry Program Exchange, Vol. 10, No. 86 (1990).
[38] Schmidt, M. W., Baldridge, K. K., Boatz, J. A., Elbert, S. T., Gordon, M. S., Jensen, J. J, Koseki, S., Matsunaga, N., Nguyen, K. A., Su, S., Windus, T. L., Dupuis, M., Montgomery, J. A., “General atomic and molecular electronic structure system”, J. Comput. Chem., Vol. 14, 1347-1363 (1993).

Partial charges may also be assigned to each covalently bound or other electrically neutral atoms of a molecule as per an molecular mechanics all-atom force field, especially for macromolecules such as proteins, DNA/RNA, etc. Such force fields may be used to assign various other atomic, bond, and/or other chemical or physical descriptors associated with components of molecules including, but not limited to, such items as vdW radii, solvation dependent parameters, and equilibrium bond constants. Examples of such force fields include AMBER [39][40], OPLS [41], MMFF [42], CHARMM [43], and the general-purpose Tripos force-field [44] of Clark et al.
[39] Pearlman, D. A., Case, D. A., Caldwell, J. C., Ross, W. S., Cheatham III, T. E., Ferguson, D. M., Seibel, G. L., Singh, U. C., Weiner, P., Kollman, P. A. AMBER 4.1, University of California, San Francisco (1995).
[40] Cornell, W. D., Cieplak, P., Bayl), C. I., Goulg, I. R., Merz, K. M., Ferguson, D. M., Spellmeyer, D. C., Fox, T., Caldwell, J. W., Kollman, P. A., “A second-generation force field for the simulation of proteins, nucleic acids, and organic molecules”, J. American Chemical Society, Vol. 117, 5179-5197 (1995).
[41] Jorgensen, W. L., & Tirado-Rives, J., J. American Chemical Society, Vol. 110, 1657-1666 (1988).
[42] Halgren, T. A., “Merck Molecular Force Field I. Basis, Form, Scope, Parameterization, and Performance of MMFF94”, J. Comp. Chem., Vol. 17, 490-519 (1996).
[43] Brooks, B. R., Bruccoleri, R. E., Olafson, B. D., States, D. J., Swaminathan, S. and Karplus, M., “CHARMM: A Program for Macromolecular Energy, Minimization, and Dynamics Calculations”, J. Comp. Chem., Vol. 4, 187-217 (1983).
[44] Clark, M., Cramer, R. D., Opdenbosch, N. V., “Validation of the General Purpose Tripos 5.2 Force Field”, J. Comp. Chem., Vol. 10, 982-1012 (1989).

There are multiple examples of prior art in the field of active site prediction. In such cases, the goal of theoretical active site prediction methods is to avoid labor-intensive, time-consuming and/or expensive experimental processes, in order to rationalize and make more efficient the target validation process. Thus, accurate and efficient predictions of active sites of biopolymers are of great interest for the biotechnology and pharmaceutical industry.

Examples of prediction methods based on structural or sequence homology as it pertains to protein functional motifs include:
[45] Chakrabarti, R., Klibanov, A. M., Friesner, R., “Computational Prediction of native protein ligand-binding and enzyme active-site sequences”, PNAS, Vol. 102, no. 29, 10153-10158 (2005).
[46] Zvelebil, M. J., Barton, G. J., Taylor, W. R., Sternberg, M. J., “Prediction of protein secondary structures and active sites using the alignment of homologous sequences”, J. Mol. Biol, Vol. 195, no. 4, 957-961 (1987).

An alternative example method based on statistical analysis and theoretical microscopic titration curves (THEMATICS) is:
[47] Shehadi, I. A., Abyzov, A., Uzun, A., Wei, Y., Murga, L. F., Ilyin, V., Ondrechen, M. J., “Active site prediction for comparative model structures with Thematics”, J. Bioinform Comput Bio, Vol. 3, no. 1, 127-143 (2005). 89).

Yet another alternative example method based on use of an interaction map of a vdW probe rolled over the surface of the protein is:
[48] Laurie, A. T. R., Jackson, R. M., “Q-SiteFinder: an energy-based method for the prediction of protein-ligand binding sites”, Bioinformatics, Vol. 21, no. 9, 1908-1916 (2005).

There are multiple examples of academic or commercial software tools that involve active site prediction on proteins, such as:

The PROSITE program maintained by the Swiss Institute of Bioinformatics (hosted on the Internet at http://ca.expasy.ch/prosite) is a freely available public domain tool based on application of various search criteria for functional protein motifs and substructures.

The SuperStar™ program, maintained by the Cambridge Crystallographic Data Centre (CDCC) (hosted on the Internet at http://www.ccdc.cam.ac.uk/prods/superstar/index.html) is a program for generating maps of interaction sites in proteins using experimental information about inter-molecular interactions. The interactions are therefore fully knowledge-based and not ab initio generated. They can be obtained from the CCDC interaction database that contains information about non-bonded interactions from both the Cambridge Structural Database (CSD) and the Protein Data Bank (PDB).

The LigandFit™ program is a part of the Cerius² software platform from Accelrys of San Diego, Calif. The LigandFit™ program is most commonly used as a docking tool for the analysis of receptor-ligand interactions. However, a sub-module of the LigandFit™ program attempts to automatically predict potential active sites for an input protein. The sub-module uses a cavity detection algorithm for detecting invaginations in the protein as candidate active site regions.

However, none of these tools or aforementioned methods offers a robust, accurate solution to the problem of active site detection when a) the active sites are not well characterized by known “training set” motifs or sequence-based alignments, b) there is a lack of experimental (holo) structures involving known or natural binders, or c) when the active sites in question are shallow in nature. Moreover, the aforementioned methods or tools often fail to find secondary active sites not directly associated with the primary well characterized active site.

### BRIEF SUMMARY OF THE INVENTION

Aspects of the present invention relate to a method and apparatus for the estimation of locations of one or more active sites of a target biopolymer based on collective analysis of molecular combinations featuring two or more molecular subsets, wherein one of the molecular subsets are from a plurality of molecular subsets selected from a molecule library while the other is from the target biopolymer.

In one embodiment, a computerized estimator/analyzer analyzes each molecular combination to determine a plurality of molecular configurations with high relative affinity. The collective set of such configurations along with their attendant scores or predicted affinity values are then jointly analyzed via statistical and/or clustering techniques in order to define interaction loci on the molecular surface of the target biopolymer molecular subset. Further analysis of the interaction loci then produces an estimation of the location of one or more active sites on the target biopolymer.

Such a system can be used in conjunction with features described in Kita II and Kita I relating to the computation of shape complementarity and electrostatic affinity based on basis expansions of the molecular surfaces and the charge distributions and electrostatic potential fields of two or more molecular subsets in a molecular combination.

### BRIEF DESCRIPTION OF THE DRAWINGS

A more complex appreciation of the invention and many of the advantages thereof will be readily obtained, as the same becomes better understood by references to the detailed description when considered in connection with the accompanying drawings, wherein:

FIGS. 1A and 1B illustrate a molecular combination featuring a protein and a small molecule ligand;

FIG. 2 is a block diagram view of an embodiment of a system that utilizes aspects of the present invention to perform active site identification.

FIG. 3 shows a set of interaction loci found for the protein showcased in FIGS. 1A and 1B.

FIG. 4 is a flow diagram of an exemplary method for estimating the location of one or more active sites of target biopolymer molecular subset using technologies described in Kita I and Kita II;

FIGS. 5A and 5B show illustrations of two molecular subsets in two different configurations that are examples of configurations that might be explored in accordance with embodiments of the present invention.

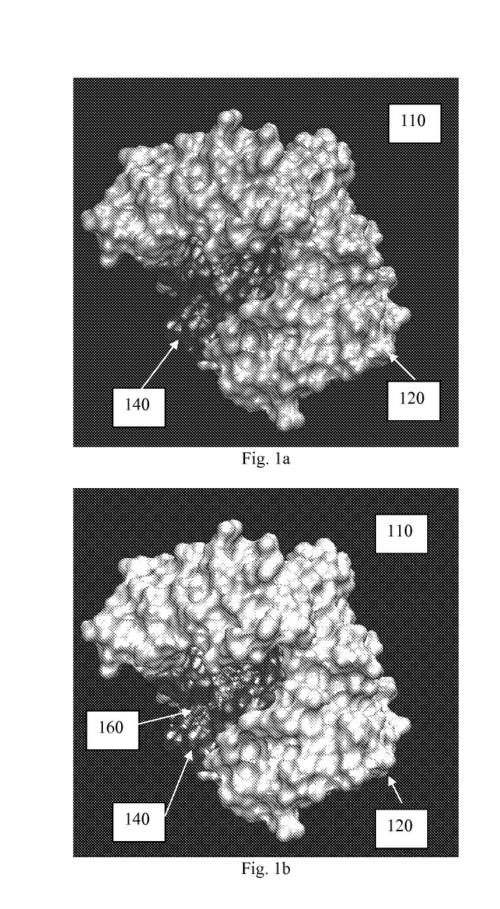

### DETAILED DESCRIPTION OF THE INVENTION

In general, the present invention relates to an efficient computational method for estimating the location of one or more active sites on a target biopolymer based on the collective analysis of molecular combinations featuring two or more molecular subsets, wherein one of the molecular subsets are from a plurality of molecular subsets selected from a molecule library while the other is from the target biopolymer. In one aspect, the invention analyzes each potential molecular combination to determine a plurality of molecular configurations with high relative affinity. The collective set of such configurations along with their attendant scores or predicted affinity values may then be combined across the library and subjected to a statistical and/or clustering analysis. Based on the results of this analysis, a plurality of interaction loci is mapped out on the molecular surface of the target biopolymer. These interaction loci can then be further analyzed to identify the locations of one or more active sites of the target biopolymer. Detailed considerations will also be discussed wherein the analysis of each molecular combination utilizes efficient rigid-body docking methods, and in particular the inventions disclosed in Kita I and Kita II.

The present invention has many applications, as will be apparent after reading this disclosure. In describing an embodiment of a computational system according to the present invention, only a few of the possible variations are described. Other applications and variations will be apparent to one of ordinary skill in the art, so the invention should not be construed as narrowly as the examples, but rather in accordance with the appended claims.

Embodiments of the invention will now be described, by way of example, not limitation. It is to be understood that the invention is of broad utility and may be used in many different contexts.

A molecular subset is a whole or parts of the components of a molecule, where the components can be single atoms or bonds, groups of atoms and/or bonds, amino acid residues, nucleotides, etc. A molecular subset might include a molecule, a part of a molecule, a chemical compound composed of one or more molecules (or other bio-reactive agents), a protein, one or more subsets or domains of a protein, a nucleic acid, one or more peptides, or one or more oligonucleotides. In another embodiment of the present invention, a molecular subset may also include one or more ions, individual atoms, or whole or parts of other simple molecules such as salts, gas molecules, water molecules, radicals, or even organic compounds like alcohols, esters, ketones, simple sugars, etc. In yet another embodiment, the molecular subset may also include organic molecules, residues, nucleotides, carbohydrates, inorganic molecules, and other chemically active items including synthetic, medicinal, drug-like, or natural compounds.

In yet another embodiment, the molecular subset may already be bound or attached to the target through one or more covalent bonds. In another embodiment the molecular subset may in fact include one or more structural components of the target, such as secondary structure elements that make-up a tertiary structure of a protein or sub-units of a protein quaternary structure. In another embodiment the molecular subset may include one or more portions of a target molecule, such as protein domains that include the whole or part of an active site, one or more spatially connected subsets of the protein structure that are selected based on proximity to one or more protein residues, or even disconnected protein subsets that feature catalytic or other surface residues that are of interest for various molecular interactions. In another embodiment, the molecular subset may include the whole of or part of an existing molecular complex, resulting in a molecular combination involving a target biopolymer with more than one binder interacting at an alternative binding site such as for example, an activated protein or an allosterically bound protein.

A molecular combination (or combination) is a collection of two or more molecular subsets that may potentially bind, form a molecular complex, or otherwise interact with one another. A combination specifies at the very least the identities of the two or more interacting molecular subsets.

A molecular pose is the geometric state of a molecular subset described by its position and orientation within the context of a prescribed coordinate system. A molecular configuration (or configuration) of a molecular combination represents the joint poses of all constituent molecular subsets of a molecular combination. Different configurations are denoted by different relative positions and orientations of the molecular subsets with respect to one another. Coordinate transformations that do not change the relative position or orientation of constituent molecular subsets will not result in different configurations.

For the purposes of the invention different configurations of a molecular combination are obtained by the application of rigid body transformations, including relative translation and rotation, to one or more molecular subsets. For the purposes of the invention, such rigid body transformations are expected to preserve the conformational structure, as well as the stereochemistry, of each molecular subset. In regards to the present invention it is contemplated that when analyzing distinct conformations or stereoisomers of a molecular subset, each distinct conformation or stereoisomer will appear in a distinct molecular combination, each with its own attendant analysis. In this way, molecular combinations featuring flexible molecular subsets may be better analyzed using the invention based on consideration of multiple combinations comprising distinct conformations and/or stereoisomers.

In many of the forthcoming examples and explanations, the molecular combination will represent the typical scenario of two molecular subsets where a ligand biomolecule (first molecular subset) interacts with a target biomolecule (usually a biopolymer; second molecular subset). Thus in regards to the present invention, analysis of a molecular combination may seek to determine whether, in what fashion (i.e., binding mode), and/or to what degree, a ligand will interact with a target molecule based on computational modeling of binding affinity of one or more configurations. A detailed discussion of the concept of binding affinity will be forthcoming in the description, and more specifically how it can be related to the concepts of shape complementarity and electrostatic affinity. It should be understood that, unless otherwise indicated, such examples and explanations could more generally apply to molecular combinations wherein more than two molecular subsets bind or interact with one another, representing the whole of, or portion(s) of, one or more target molecules and/or one or more ligands.

As an example, in one embodiment of the present invention, the molecular combination may represent a target interacting with a ligand (i.e., target-ligand pair) where one molecular subset is from the protein and the other the ligand. In a further embodiment, the molecular combination may represent a target-ligand pair where one molecular subset is the entire ligand biomolecule but the other molecular subset is a portion of a target biopolymer containing one or more relevant active sites.

In yet another embodiment, the molecular combination may feature more than two molecular subsets, one representing a target (whole or part) and the other two correspond to two distinct ligands interacting with the same target at the same time, such as in the case of competitive thermodynamic equilibrium between a possible inhibitor and a natural binder of a protein. In yet another embodiment the previous example may be turned around such that the molecular combination features two target molecules in competition with one ligand biomolecule.

As another example, in one embodiment the molecular combination may represent a protein-protein interaction in which there are two molecular subsets, each representing the whole or a relevant portion of one protein. In a further embodiment, the molecular combinations may also represent a protein-protein interaction, but now with potentially more than two molecular subsets, each representing an appropriate protein domain.

As a further example, the molecular combination may feature two molecular subsets representing a target-ligand pair but also additional molecular subsets representing other atoms or molecules (hetero-atoms or hetero-molecules) relevant to the interaction, such as, but not limited to, one or more catalytic or structural metal ions, one or more ordered, bound, or structural water molecules, one or more salt molecules, or even other molecules such as various lipids, carbohydrates, acids, bases, mRNA, ATP/ADP, etc. In yet another embodiment, the molecular combination may feature two molecular subsets representing a target-ligand pair but also one or more added molecular subsets representing a whole or portion of a cell membrane, such as a section of a lipid bi-layer, nuclear membrane, etc., or a whole or portion of an organelle such as a mitochondrion, a ribosome, endoplasmic reticulum, etc.

In another embodiment, the molecular combination may feature two or more molecular subsets representing protein chains or sub-units interacting non-covalently as per a quaternary protein structure. In another embodiment, the molecular combination may feature two or more molecular subsets representing protein secondary structure elements interacting as per a tertiary structure of a polypeptide chain, induced for example by protein folding or mutagenesis.

In many of the forthcoming examples and explanations, the molecular combination will represent the typical scenario of a target-ligand pair. As already mentioned in regards to the present invention, an analysis of a molecular combination may seek to determine whether, in what fashion, and/or to what degree or with what likelihood, a ligand will interact with a target molecule based on computations of binding affinity. In another embodiment, the analysis may involve a plurality of molecular combinations, each corresponding to a different ligand, selected, for example, from a molecule library (virtual or otherwise), in combination with the same target molecule, in order to find one or more ligands that demonstrate high binding affinity with the target, and are therefore likely to bind or otherwise react with the target. In such cases, it may be necessary to assign a score or ranking to each analyzed molecular combination based on the estimated binding affinity across a set of different configurations for each combination, in order to achieve relative comparison of relevant predicted bioactivity.

In such a scenario where each target-ligand pair is an individual combination, and if there are N ligands to be tested against one target, then there will be N distinct molecular combinations involved in the analysis. For sufficiently large molecule libraries, it may be necessary to analyze millions or more potential molecular combinations for a single target protein. In yet another embodiment, the analysis may be reversed and the plurality of molecular combinations represents a plurality of target molecules, each in combination with the same ligand biomolecule in the same environment. In other embodiments, the molecular combinations may represent multiple ligands and/or targets reacting simultaneously, i.e., more than just a target-ligand pair, and may also include various heteroatoms or molecules as previously discussed.

In general, embodiments of the present invention provide an efficient computational method for estimating locations of one or more active sites on a target biopolymer by analyzing collective results for modeling the likelihood and nature of molecular combinations between a library of molecules and the target biopolymer. These methods can be based on analyzing configurations for each molecular combination involving the molecule library and the target biopolymer. The configuration results, with attendant scoring metrics, are then combined across the library and subjected to a statistical and/or clustering analysis. Based on the results of this analysis, interaction loci are mapped out on the molecular surface of the target biopolymer. These interaction loci can then be further analyzed to identify the locations of one or more active sites of the target biopolymer. The analysis of each molecular combination, might then involve efficient rigid-body docking methods, such as those shown in Kita I and Kita II.

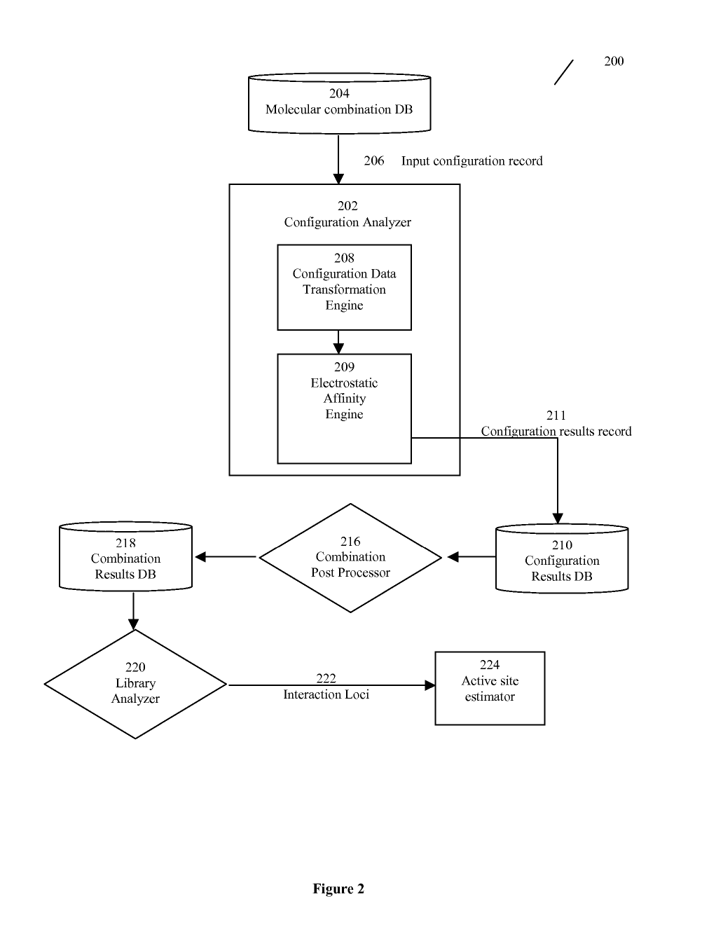

FIG. 2 illustrates an active site prediction system 200 for the estimation of the location of active site(s) of a biopolymer based on the analysis of molecular combinations associated with a molecule library and a target biopolymer wherein said analysis includes computations of an affinity function across a set of sampled molecular configurations for the molecular combination. As shown, a configuration analyzer 202 receives one or more input (or reference) configuration records 206, including relevant structural, chemical, and physical data associated with input structures for a pair of molecular subsets from an input molecular combination database 204. The configuration analyzer 202 comprises a configuration data transformation engine 208 and an affinity function engine 209. Results from the configuration analyzer 202 are output as configuration results records 211 to a configuration results database 210.

The active site prediction system 200 can provide an efficient analysis of a collection of molecular combinations via computations of an affinity function over a set of sampled molecular configurations. In some embodiments, the aforementioned analysis of each molecular combination may include, but is not limited to, prediction of likelihood of formation of a potential molecular complex, or a proxy thereof, the estimation of the binding affinity between molecular subsets in the molecular combination, the prediction of the binding mode (or even additional alternative modes) for the molecular combination with an attendant affinity function value, or a rank prioritization metric or (affinity) score based on affinity with the target biopolymer molecular subset across sampled configurations of the combination, or one of the many other usages associated with computational target-ligand docking. Herein, an “affinity score” for a configuration refers to the affinity function value or metric used to assess or analyze individual molecular configurations. There will be more discussion related to various embodiments of the affinity function used in the invention further on in the description.

As the total possible number of configurations may be enormous, the configuration analyzer 202 may sample a subset of the possible configurations during the analysis procedure according to an appropriate sampling scheme or protocol. In one embodiment, as part of the configuration analysis of a molecular combination, the configuration data transformation engine 208 provides for performing a dense search in the configurational space of two or more molecular subsets having rigid bodies, that is, assessing relative orientations and translations of the constituent molecular subsets.

In another embodiment, the method can also be used in conjunction with a process for generating likely yet distinct conformations of one or both molecular subsets, in order to better analyze those molecular combinations where one or both of the molecular subsets are flexible.

In yet another embodiment, the configuration data transformation engine 208 employs methods more appropriate for dense sampling of molecular configurations when one or both of the molecular subsets is structurally flexible. In one embodiment, this may be in the form of a stochastic optimization search that explores the joint configurational space of the two molecular subsets according to a stochastic paradigm such as that found in simulated annealing, Monte Carlo, or genetic algorithm docking methods or similar methods.

In a typical operation, in order to obtain an accurate estimation of the location of one or more active sites for a target biopolymer, many molecular combinations, each featuring many different configurations, may be analyzed. Note that the sampled set of molecular configurations may be very large (e.g., millions or even possibly billions of configurations per combination). An affinity score is generated for each sampled configuration and the results for one or more configurations are recorded in a storage medium.

Each molecular combination may then be further analyzed by comparative examination of the set of stamped configuration results including the corresponding computed affinity scores. Once the cycle of computation is complete for one molecular combination, modeling of the next molecular combination may ensue. Alternatively, in some embodiments of the active site prediction system 200, multiple molecular combinations may be modeled in parallel. In some embodiments, during modeling of a molecular combination, more than one configuration may be processed in parallel as opposed to simply in sequence. Potentially millions of such molecular combinations may be analyzed in order to determine one or more active sites on the target biopolymer molecular subset. Thus, there may be certain practical limitations on the amount of data to be stored for each molecular combination analysis.

In one embodiment, active site prediction system 200 may be implemented on a dedicated microprocessor, ASIC, or FPGA. In another embodiment, active site prediction system 200 may be implemented on an electronic or system board featuring multiple microprocessors, ASICs, or FPGAs. In yet another embodiment, active site prediction system 200 may be implemented on or across multiple circuit boards housed in one or more electronic devices. In yet another embodiment, active site prediction system 200 may be implemented across multiple devices containing one or more microprocessors, ASICs, or FPGAs on one or more electronic boards and the devices connected across a network.

In some embodiments, active site prediction system 200 may also include one or more storage media devices for the storage of various, required data elements used in or produced by the analyses. Alternatively, in some other embodiments, some or all of the storage media devices may be externally located but networked or otherwise connected to the active site prediction system 200. Examples of external storage media devices may include one or more database servers or file systems. In some embodiments involving implementations featuring one or more circuit boards, the active site prediction system 200 may also include one or more software processing components in order to assist the computational process. Alternatively, in some other embodiments, some or all of the software processing components may be externally located but networked or otherwise connected to the active site prediction system 200.

In some embodiments, once analysis of a molecular combination is completed (i.e., all desired configurations assessed) a combination post-processor 216 may be used to select one or more configuration results records from configuration results database 210 in order to generate one or more either qualitative or quantitative measures for the combination, such as a combination score, a combination summary, a combination grade, etc. The resultant combination measures are then stored in a combination results database 218. In one embodiment, the combination measure may reflect the configuration record stored in configuration results database 210 with the best-observed configuration affinity score. In another embodiment, multiple configurations with high affinity are submitted to the combination post-processor 216 and a set of combination measures written to the combination results database 218. In another embodiment, the selection of multiple configurations for use by the combination post-processor 216 may involve one or more thresholds or other decision-based criteria.

In a further embodiment, the combination measures output to the combination results database 218 are based on various statistical and/or clustering analysis of a (possibly large) sampling of configuration results records stored in database 210. As used herein, the term “cluster analysis” or “clustering analysis” generally refers to a multivariate analysis that seeks to organize information about variables so that relatively homogeneous groups, or “clusters,” can be formed. In one embodiment, the clusters formed with this family of methods should be highly, internally homogenous (e.g., with members from same cluster being similar to one another) and highly, externally heterogeneous (e.g., members of each cluster being different from members of other clusters). In another embodiment, the selection sampling itself may be based on statistical methods (e.g. principal component analysis, multi-dimensional clustering, multivariate regression, etc.) or on pattern-matching or training methods (e.g. neural networks, support vector machines, etc.)

In another embodiment, the combination post-processor 216 may be applied dynamically (i.e., on-the-fly) to the configuration results database 210 in conjunction with the analysis of the molecular combination as configuration results records become available. In yet another embodiment, the combination post-processor 216 may be used to rank different configurations in order to store a sorted list of either all or a subset of the configurations stored in configuration results database 210 that are associated with the combination in question. In yet other embodiments, once the final combination results records, reflecting the complete analysis of the molecular combination by the configuration analyzer 202, have been stored in database 218, some or all of the configuration records in configuration results database 210 may be removed or deleted in order to conserve storage in the context of a library-wide analysis involving possibly many different molecular combinations. Alternatively, some form of garbage collection may be used in other embodiments to dynamically remove low affinity or otherwise “poor” configuration results records from configuration results database 210.

In one embodiment, the molecular combination record database 204 may comprise one or more molecule records databases (e.g. flat file, relational, object oriented, etc.) or file systems and the configuration analyzer 202 receives an input molecule record corresponding to an input structure for each molecular subset of the combination. In another embodiment, when modeling molecular combinations, the molecular combination record database 204 is replaced by an input target record database and an input ligand (or drug candidate) record database. In a further embodiment, the input target molecular records may be based on either experimentally derived (e.g. X-ray crystallography, NMR, etc.), energy minimized, or model-built 3-D protein structures. In another embodiment, the input ligand molecular records may reflect energy minimized or randomized 3-D structures or other 3-D structures converted from a 2-D chemical representation, or even a sampling of low energy conformers of the ligand in isolation. In yet another embodiment, the input ligand molecular records may correspond to naturally existing compounds or even to virtually generated compounds, which may or may not be synthesizable.

Examples of file formats for input molecule record(s) that may be part of an input configuration record submitted to configuration analyzer 202 include, but are not limited to, the PDB, mol2, CHIME, and CHARMM file formats. For the sake of brevity, we refer the reader to the relevant figures in Kita I and Kita II for additional examples.

In one embodiment the configuration data transformation engine 208 may directly transform one or more input molecular configurations into one or more other new configurations by application of various rigid body transformations. In other embodiments, the configuration data transformation engine 208 may instead use features described in Kita I and Kita II for applying rigid body transformations to sets of basis expansion coefficients representing molecular surfaces, and charge density and electrostatic potential functions associated with reference poses for each molecular subset. In some embodiments, the set of configurations visited during the course of an analysis of a molecular combination may be determined according to a schedule or sampling scheme specified in accordance with a search of the permitted configuration space for the molecular combination.

In some embodiments, the configuration data transformation engine 208 may produce new configurations sequentially and feed them one by one to the affinity function engine 209. In another embodiment, the configuration data transformation engine 208 may instead produce new configurations in parallel and submit them to the affinity function engine 209 in concert.

The affinity function engine 209 is responsible for generating an affinity score or equivalent measure for each sampled configuration of a molecular combination. As described above, an affinity score can be any measure quantitative or qualitative in nature that reflects or correlates in some manner to the binding affinity of a molecular combination.

In one embodiment, the affinity function engine 209 makes use of features described in Kita I and Kita II to efficiently compute an augmented (affinity) score via shape complementarity and electrostatic affinity for each configuration based on use of basis expansions and rigid body transformations of molecular surfaces and charge density and electrostatic potential functions associated with each pair of molecular subsets. The affinity function engine 209 may also include one or more storage components for data specific to the computations of affinity.

In some embodiments, the configuration results records 211 may include a quantitative measure related to the affinity evaluated for each configuration. In one embodiment, this may be a score. In another embodiment, this may be a probability. In other embodiments, the configuration results records 211 may include a qualitative measure related to the affinity evaluated for the configuration. In one embodiment, this may be a grade. In another embodiment this may be a categorization (i.e., poor, weak, strong, etc.). In yet another embodiment this may be a simple pass-fail measure.

In many embodiments, the configuration results records 211 may also include information used to specify the identity and/or nature of configuration corresponding to a given affinity score. In addition to the identity of the interacting molecular subsets, there may be a need to annotate or otherwise represent the geometrical state of the configuration.

For example, in the context of rigid-body docking analysis, typically this may be achieved by storing the parameters of the rigid body transformation used to generate the configuration from an input or reference configuration.

In some embodiments, the configuration results database 210 may utilize various selection criteria in order to accept a subset of configuration records 211. In one embodiment, the selection criteria may be predicated on passing of a threshold or other decision mechanism based on one or more affinity measures or affinity scores. In other embodiments, such analysis may in part take place in the combination post-processor 216.

In yet another embodiment, the selection criteria used by the configuration results database 210 may also be based on various statistical analysis of a number of different configuration results records already stored in database 210, including, but not limited to, principal component analysis, multi-dimensional clustering, Bayesian filters, multivariate regression analysis, etc. In yet another embodiment, the selection criteria may be based on various pattern matching analysis of a number of different configuration results records stored in database 210, including, but not limited to, use of neural networks, support vector machines, hidden Markov models, etc.

Once the combination results have been generated for a plurality of combinations involving molecular subsets from the molecule library and the target biopolymer, a library analyzer 220 can begin analysis of the combination results. In one embodiment, such analysis is performed only once all molecular combinations involving the library have been generated. In other embodiments, the analysis is initiated once a sufficient number of combination results have been generated.

One task of the library analyzer 220 is to identify interaction loci by jointly comparing and analyzing the combination results (including the score and the resultant configurations as discussed above) across the library. In one aspect, 1 the interaction loci are associated with a portion or region of the target protein that are in the proximity to portions of one or more molecular subsets from the library as they are placed in the combination results records comprising one or more favorable configurations. In one embodiment, after suitable normalization for difference in size and structural degrees of freedom between molecular subsets from the molecule library, the sets of combination results are rank prioritized and only a top percentage of ranked results are retained for identifying the interaction loci. In another embodiment, only those combination results above a pre-determined threshold are retained for further analysis.

The passing sets of combination results are then examined to determine a plurality of spatial locations on or near the molecular surface for which there is strong consensus or overlap amongst multiple favorable configurations from the passing combinations. In one embodiment, such analysis only involves the most favorable or highest-scoring configuration for each passing combination. In another embodiment, only structurally distinct configurations with high relative score (across the library) are used. In another embodiment, corresponding affinity scores are used to assign weights or probabilities to the spatial locations.

There can be many alternative methods for constructing a consensus or overlap based on the passing molecular combination results. In the end, library analyzer 220 preferably generates a plurality of interaction loci 222 that will be used by the active site estimator 224.

In one embodiment, a quantitative or qualitative score or measure is associated with each interaction locus that reflects a likelihood of the locus being in or near an active site. In some embodiments, this interaction measure is normalized to account for differing representations of chemical moieties or classes in the library in order to prevent bias.

A preferred embodiment is described further below. That preferred embodiment involves use of weighted projections of favorable configurations onto a high-resolution 3-D grid enclosing the target biopolymer molecular surface in order to build a consensus map which is then analyzed by various decision criteria to define the interaction loci.

Finally, the active site estimator 224 analyzes the plurality of input interaction loci 222 to make predictions about the locations of one or more active sites of the target biopolymer. The predictions are based on the spatial arrangement of the interaction loci as well as their interaction measures. Areas of high density of strong interaction loci would tend to suggest a high likelihood of a portion of an active site. Areas of low density of weak interaction loci would tend to suggest little likelihood of the region being in or near an active site. By performing a suitable statistical and/or clustering analysis, the plurality of interaction loci can be pruned to retain those interaction loci, which are of high likelihood of being associated with genuine active sites. In this way, the active site estimator 224 can map out the locations of one or more active sites of the target biopolymer.

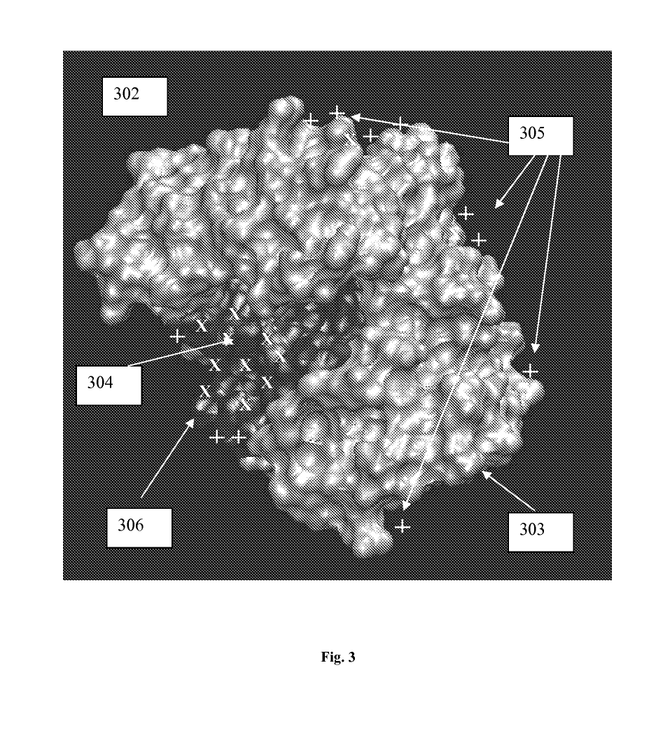

FIG. 3 is useful for understanding how interaction loci can be used to estimate the location of one or more active sites of as target biopolymer. In FIG. 3, the target biopolymer molecular subset 302 represents the human protein DHFR as illustrated previously. In FIG. 3, a molecular surface 303 is depicted for molecular subset 302. In FIG. 3, a collection of interaction loci 304 are shown as crosshairs and plus signs on the molecular surface 303. The crosshairs reflect strong interaction loci, whereas the plus signs reflect weaker loci. Region 305 is a low-density region with few interaction loci of reasonable strength. It is likely then that region 305 is not in or near an active site of human DHFR. As a counter-example, region 306 is a high-density region with many strong (i.e., bright) interaction loci and as such is very likely to be part of an active site of human DHFR.

In some embodiments, active site estimator 224 is further refined by including a machine learning protocol such as a neural net or support vector machine to train on a collection of target biopolymers with known, well-characterized active sites. The machine learning protocol is then used to better parameterize the interaction measures and to make improved judgments of active site locations based on observed patterns of putative interaction loci found in other biopolymers by incorporating important criteria that would be difficult to characterize in an ab initio manner.

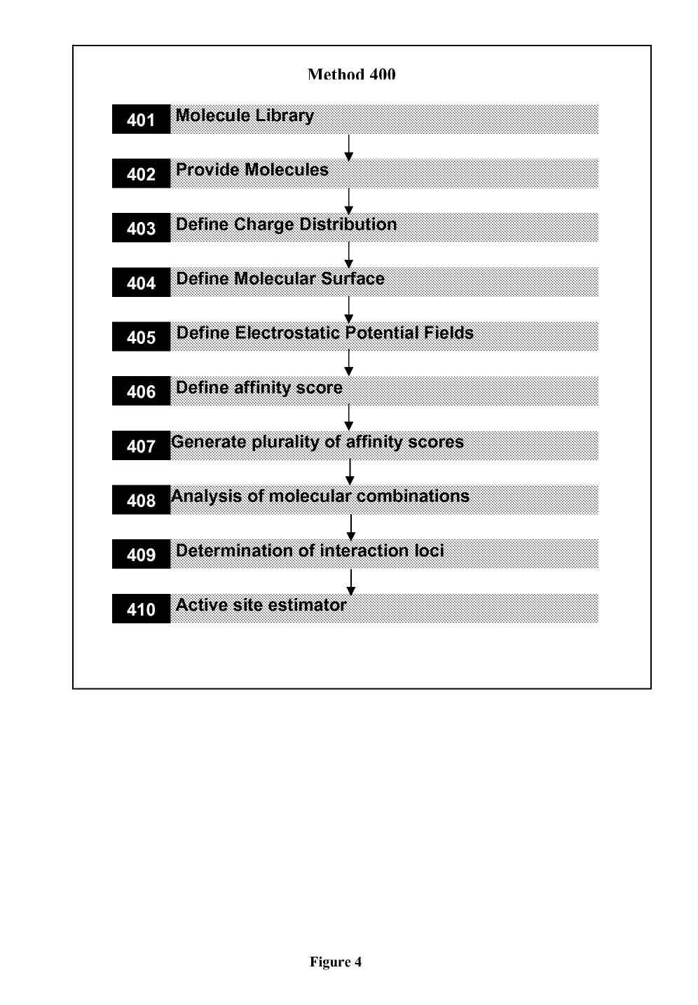

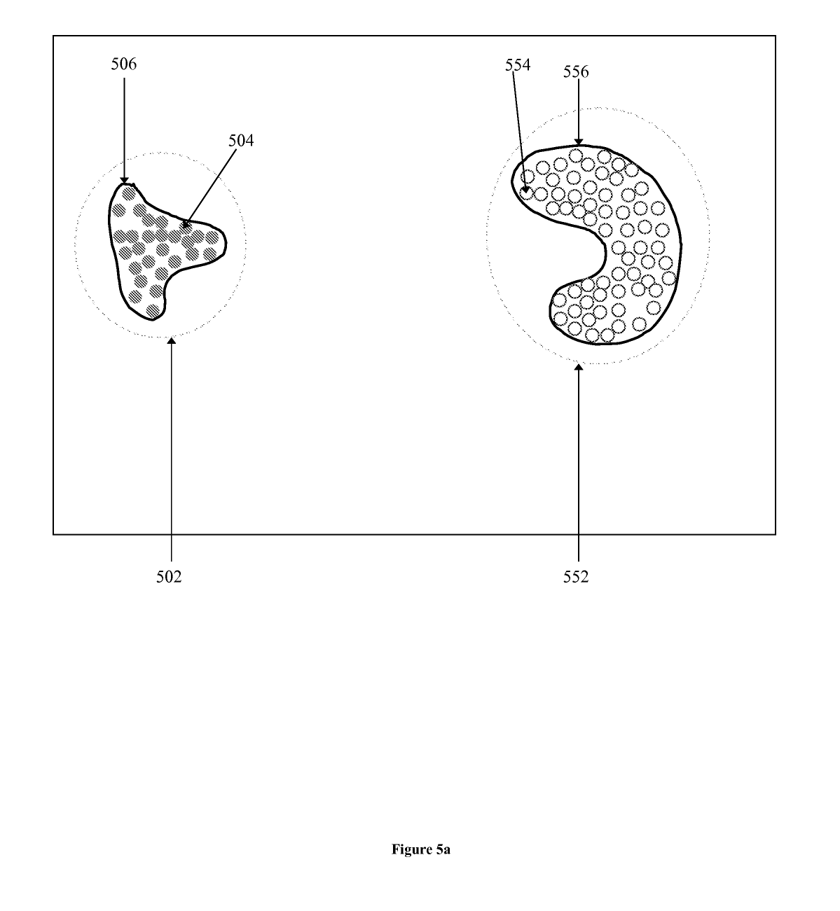

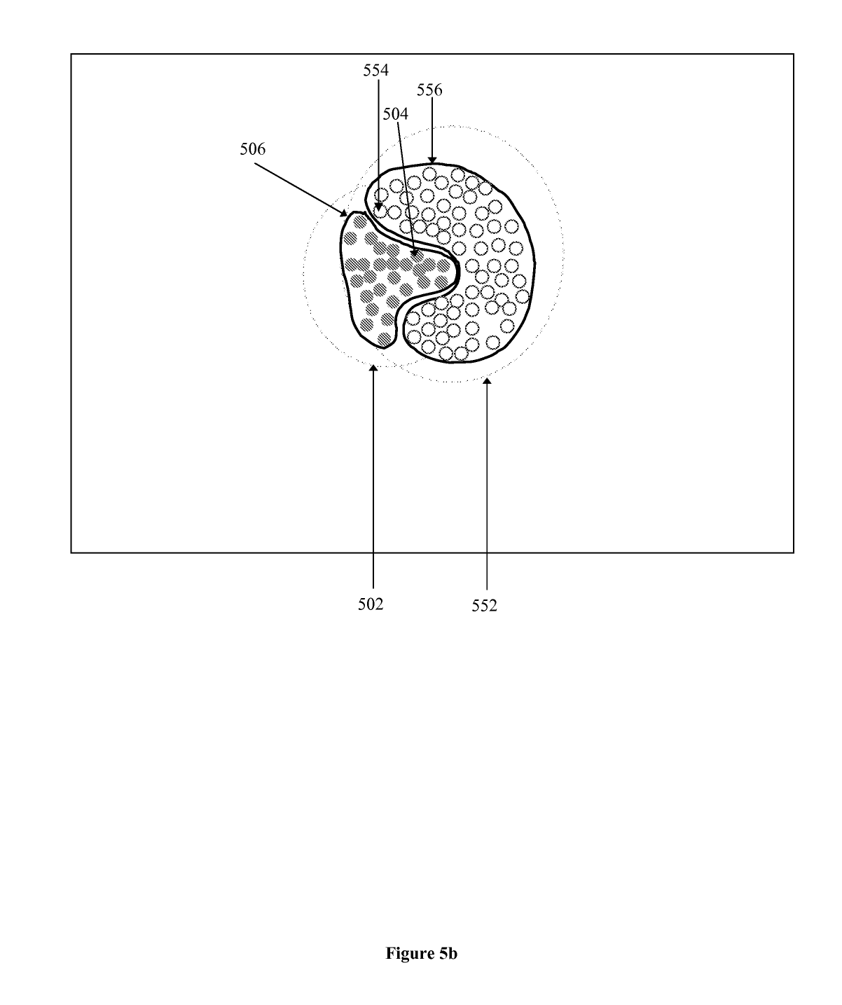

FIG. 4 is a flow diagram of an exemplary method 400 of estimating the location of one or more active sites of a target biopolymer molecular subset based on analyzing a collection of molecular combinations involving a molecule library and a target biopolymer, performed in accordance with embodiments of the present invention. Some embodiments of method 400 make use of various features described in Kita I and/or Kita II. The method 400 of FIG. 4 is also described with reference to FIGS. 5A and 5B.

As explained below, the exemplary method 400 generally involves analyzing a collection of molecular combinations by jointly assessing shape complementarity and electrostatic affinity for each combination over a set of sampled configurations as part of a dense high resolution search of rigid-body conformational space of each pair of molecular subsets, where one subset is from the target biopolymer and the other from the molecule library. Based on the resultant analysis of the molecular combinations, a set of interaction loci are identified on the molecular surface of the target biopolymer molecular subset. Each interaction locus is associated with a probability value or other interaction measure in order to reflect the consensus of favorable combinations in differing configurations, which overlap or contain the interaction locus in question. An ensuing (weighted) analysis of the spatial distribution of the interaction loci can be expected to lead to an accurate estimation of the location of one or more active sites of the target biopolymer. Embodiments of this method incorporate various combinations of hardware, software, and firmware to perform the steps described below.

In FIG. 4, in step 402, a first molecular subset 502 from a molecule library 401 and a second molecular subset 552 from a target biopolymer are provided, as shown in FIG. 5A. The molecular subsets used herein are generally stored as digital representations in a molecule library or other collection, such as the molecular combination database 204 of active site prediction system 200 in FIG. 2. Each molecular subset has a molecular shape, as illustrated in FIG. 5A, wherein the term “molecular shape” generally refers to a volumetric function representing the structure of a molecular subset comprising a plurality of atoms and bonds. Molecular subsets 502 and 552 respectively include a plurality of atoms 504 and 554 that are generally connected by chemical bonds in order to define the structure of each molecular subset. Those skilled in the art will appreciate that the molecular subsets 502 and 552 may have various shapes other than those shown in FIG. 5A.

In one embodiment, the molecular subsets from the molecule library 401 include various chemical compounds such as would be typically associated with drug discovery, i.e., small drug-like molecules. In another embodiment, molecule library 401 includes polypeptide or even proteins. In yet another embodiment, molecule library 401 includes individual residues or amino acids or other chemical sub-units of biopolymers. In yet another embodiment, the molecule library 401 includes individual entries for various chemical groups or even individual atoms. In yet another embodiment, the molecule library 401 includes various fragments or building blocks of chemical compounds. Molecule library 401 might include more than one of the sets just described. In a preferred embodiment, the molecule library might include an assortment of representative chemical compounds and informative fragments thereof.

In FIG. 4, in step 403, charge distributions are assigned to each molecular subset in accordance with various embodiments outlined in Kita I. In FIG. 4, in step 404, molecular surfaces are also assigned to each molecular subset in accordance with various embodiments outlined in Kita II. The first molecular surface is defined by recognition that some of atoms 504 are situated along a border or boundary of molecular subset 502. Such atoms are herein referred to as “surface atoms”. The surface atoms are proximal to and define a molecular surface 506 for the first molecular subset 502 based on their location. In FIG. 4, in step 405, electrostatic potential fields are also assigned to each molecular subset in accordance with various embodiments outlined in Kita I.

In FIG. 4, in step 406, an affinity function is defined. As used herein, an “affinity score” is a representation of the change in total energy of a system going from a state of two molecular subsets in isolation in an ambient environment to the potential formation of a molecular complex comprised of the two molecular subsets in close proximity at a relative orientation and position to one another and embedded in the same ambient medium. It is desired for the configuration analyzer 202 to perform a dense search in the conformational space of the two molecular subsets treated as rigid bodies, i.e., for possibly millions of relative orientations and translations of the two molecular subsets, in an efficient manner, and the “affinity score” is intended to be an accurate approximation of the relevant change in binding energy associated with each sampled molecular configuration.

In a preferred embodiment, the affinity score is the augmented score S′, as described in Kita I, which is defined as follows:
S′=S−γΔE [Eqn. 1]
Here the augmented score is a linear combination of a shape complementarity score, S, and an electrostatic affinity score, ΔE, and γ is a scalar constant, meant to weight the two scores relative to one another in the composition.

FIG. 5B shows the exact same molecular subsets as those found in FIG. 5A but now in a different molecular configuration. Now the two molecular subsets are much closer to one another and aligned in such a way that they demonstrate high shape complementarity. Assuming the electrostatic affinity is also high, then one would expect that the configuration depicted in FIG. 5B have a higher affinity score than the original configuration in FIG. 5A, i.e., the configuration of FIG. 5B is a more favorable configuration than that depicted in FIG. 5A. The system takes this observation into account.

In the course of modeling a molecular combination, a plurality of affinity scores might be computed each corresponding to a different relative position and orientation of the first and second molecular subsets 502 and 552 in the course of analyzing the sampled molecular configurations for a molecular combination. In FIG. 4, in step 407, the aforementioned plurality of affinity scores are generated by iterating over the set of sampled configurations defined by the Cartesian product of the set of sampled poses for the first molecular subset and the set of sampled poses for the second molecular subset in the molecular combination. It should be understood that the iteration over the set of sampled configurations for the molecular combination, for the purpose of generating the plurality of affinity scores, can be performed in any order.

In order to increase efficiency when there is more prior knowledge about the target biopolymer molecular subset 552, in one embodiment, the computation of affinity scores is restricted to a subset of the possible orientations of the target biopolymer molecular subset by constraining the analysis to a subset of the surface of the target biopolymer. In yet other embodiments, similar logic can be applied even when the configuration data transformation engine 208 involves much more sophisticated structural transformations than just rigid-body transforms of the molecular subsets.

The generated affinity scores and their corresponding molecular configurations are then subjected to further analysis. In one embodiment, the analysis of a molecular combination 408 is generally decided based on the particular configuration, i.e., relative position and orientation, that yields the highest affinity score. In another embodiment, the magnitude of the best score, or a top percentage of scores, or other ranked selection process, determines the results of the analysis of the molecular combination of the two molecular subsets. In another embodiment, all affinity scores below a preset numerical threshold are rejected, and only those configurations with passing scores are retained for further analysis. In yet another embodiment, the affinity scores are filtered based on an adaptive threshold dependent on observed statistics of the scores as they are generated. In one embodiment, the results are quantitatively analyzed according to certain decision criteria. In another embodiment, the decision criteria are based on a cluster analysis of the affinity scores.

An intent of the combination analysis of 408 is to determine whether a particular molecular combination is suitable to be included in further downstream analysis when determining interaction loci to help identify one or more active sites of the target biopolymer. In one embodiment, the combination analysis of 408 applies a statistical analysis of the score magnitudes of passing configurations, as well as multi-dimensional clustering of the relative position and orientation coordinates, to determine a set of representative structural information or modes for the combination. In other embodiments, the combination analysis may attempt to normalize for differences in the size and conformational degrees of freedom between differing molecular subsets across the library to reduce biasing. Whatever the precise embodiment used, the passing molecular combinations are then used in step 409, for the determination of interaction loci.

In a preferred embodiment, one or more high affinity but structurally distinct configurations are retained for each passing molecular combination. Each configuration is then projected onto a 3-D grid enclosing the molecular surface of the target biopolymer. In one embodiment, an occupancy counter is assigned to each cell of the grid and is incremented each time a distinct favorable configuration overlaps the grid cell with one of its constituent atoms or bonds. In another embodiment, only heavy atoms are used to determine the grid cell occupancy. In another embodiment, only atoms and bonds in core components or scaffold groups are involved in the occupancy determination. In yet another embodiment, only certain species of atoms or atoms in certain types of chemical moieties or groups are included in the occupancy determination process. It should be understood that some data points can be discarded as a form of filtering.

In one embodiment, each passing molecular combination can contribute at most N favorable structurally distinct configurations for the occupancy determination. In yet another embodiment, only the single most favorable configuration is used. In yet another embodiment, all structurally distinct favorable configurations above a pre-determined numerical threshold are included. Other selecting rules can be used instead.

In another embodiment the occupancy counter is replaced by a numeric sum or weight where the amount contributed by the overlap of a given configuration is scaled by the affinity score for the configuration. In another embodiment, the weight is scaled by a factor reflecting the molecular subsets' structural and chemical diversity within molecule library 401. In a preferred embodiment, the occupancy sum is a weighted average of the affinity scores for all configurations from all passing combinations that overlap said grid cell. In this way, it may be possible to reduce biases from overly large representations of certain types or classes of compounds in the molecule library and to better normalize the frequency of occurrence of certain compounds in such a library. At the same time, the weighted average may reflect the inherent affinity of the molecular combinations. In another embodiment, only those grid cells within a certain closest distance of the molecular surface of the target biopolymer molecular subset are considered.

Once the list of all passing molecular combinations has been fully processed and the 3-D occupancy grid completed, then an analysis of the grid map is initiated in order to define interaction loci. In one embodiment to reduce spurious intensity values at grid cell locations, a smoothing filter is applied to the grid in order to remove artificial discontinuities and other potential artifacts that would adversely affect the ensuing analysis. In another embodiment, the grid is then scanned to find all grid cells with a minimally sufficient value. Each such cell then defines an interaction locus. In another embodiment, an interaction measure is further defined as a function of the grid cell occupancy value.

There are many possible alternative embodiments for selecting the interaction loci based on the aforementioned occupancy grid. In one embodiment, this is the spatial center of the grid cell. In another embodiment, each interaction locus is a volumetric region such as the entire grid cell. In yet another embodiment, the interaction locus may include neighboring grid cells, for example a 2×2×2 sub-grid. In another embodiment, all interaction loci must be at least a minimum distance apart either in terms of real distance units or in terms of grid cell distances.

In a preferred embodiment, a greedy method is used wherein the maximum scoring grid cell is assigned to be the first interaction loci, then the grid is further scanned for the next highest scoring grid cell that is sufficiently far away from the first interaction locus. This grid cell becomes the second interaction locus and the method is applied again in order to find the next highest scoring grid cell that is minimally far enough away from both of the first two interaction loci. This continues until a search over all passing grid cells that are spaced far enough apart have been exhausted.

Once the interaction loci (with attendant interaction measures) have been determined in step 409, they are then subjected to a final analysis by the active site estimator 410. In one embodiment, a spatial point is considered to be in or near an active site if the site estimator finds a minimally sufficient number of interaction loci within the confines of a small sphere centered on the point in question. In another embodiment, this result is scaled based also on the intensity of the interaction measures within said spherical volume. In yet another embodiment, interaction loci are clustered into different spatial regions or domains and then an aggregate measure applied across each region to determine if it is in or near one of the putative active sites of the target biopolymer molecular subset.

In a preferred embodiment, a machine learning protocol based on a support vector machine or other adaptive learning method is used to determine whether or not a given location on the molecular surface of the target biopolymer is in or near an active site by examining its proximity to interaction loci as well as the interaction strengths of the neighboring interaction loci. In another embodiment, the machine learning is trained on a set of target biopolymers with known, well-characterized active sites. In yet another embodiment, other complementary descriptors are also used by the machine learning protocol when making its decision, including but not limited to the degree of invagination of the localized portion of the molecular surface and/or the proximity of one or more structural or functional motifs or conserved residues that are typically associated with active sites on target biopolymers. In another embodiment, this may be combined with a post-processing pass in which the estimated active site region is smoothed out in order to reduce artificial discontinuities.

Whatever the methods employed, at the end of the process, active site estimator 410 might identify the locations of one or more active sites on the target biopolymer. However, it is entirely reasonable that no such active sites may be found based on the scope and content of the library analyzed. To address such occurrences, a large molecule library of potentially million of diverse fragments or compounds may be used in accordance with the methods and apparatus described herein. A library that is too limited in size or diversity will often render a false negative result. Also in a preferred embodiment, as previously mentioned, in order to better model high conformational flexibility of either of the pair of molecular subsets, it is possible to consider the Cartesian product of an ensemble of distinct conformational structures for the target biopolymer molecular subset and an ensemble of favorable and distinct conformers for each molecular subset from the molecular library. Such an embodiment would be of particular importance when modeling a target protein with an allosteric binding domain or a protein-protein binding domain.

It will be understood that the above described arrangements of apparatus and the method there from are merely illustrative of applications of the principles of this invention and many other embodiments and modifications may be made without departing from the spirit and scope of the invention as defined in the claims.

What is claimed is:

1. A method of estimating a location of one or more active sites on a target biopolymer molecular subset by analyzing collective results for a likelihood of molecular interaction between a collection of molecular subsets and the target biopolymer molecular subset, the method comprising:
receiving input molecular data comprising at least one of structural, physical, or chemical information for the target biopolymer and the collection of molecular subsets; defining an affinity function having a score that represents a likelihood of a molecular interaction between two molecular subsets for a given molecular configuration, wherein the affinity function includes the input molecular data;
sampling a set of molecular configurations for each of a plurality of molecular combinations, each combination involving the target biopolymer molecular subset and one of the molecular subsets from the collection;
computing a plurality of affinity scores, each associated with a sampled configuration;
analyzing the affinity scores, wherein analyzing the affinity scores includes determining one or more favorable molecular configurations associated with each molecular combination;
determining one or more interaction loci based on the favorable configurations for each sampled molecular combination, wherein an interaction locus is associated with at least one region of the target biopolymer molecular subset having a high likelihood of molecular interaction with one or more molecular subsets from the collection; and
estimating a location of one or more active sites on the target biopolymer molecular subset by analyzing the one or more interaction loci derived from the plurality of favorable configurations.

2. The method of claim 1, wherein the target biopolymer molecular subset is a portion or whole of a protein.

3. The method of claim 2, wherein the target biopolymer molecular subset includes one or more catalytic, allosteric or protein binding domains.

4. The method of claim 1, wherein the collection of molecular subsets includes chemical compounds.

5. The method of claim 1, wherein the collection of molecular subsets includes polypeptides.

6. The method of claim 1, wherein the collection of molecular subsets includes fragments of chemical compounds.

7. The method of claim 1, wherein the collection of molecular subsets includes individual residues or amino acids.

8. The method of claim 1, wherein the collection of molecular subsets includes chemical groups or individual atoms.

9. The method of claim 1, further comprising:
generating an ensemble of distinct structural conformations of the target biopolymer molecular subset; and
sampling a set of molecular configurations between each member of the target biopolymer conformational ensemble and each of the plurality of molecular subsets from the collection.

10. The method of claim 1, further comprising:
generating an ensemble of distinct favorable self-energy conformations for each molecular subset in the collection; and
sampling a set of molecular configurations between the target biopolymer molecular subset and each member of the conformational ensemble for each molecular subset in the collection.

11. The method of claim 1, wherein the interaction loci are restricted to lie within a certain distance of a molecular surface of the target biopolymer molecular subset.

12. The method of claim 1, wherein the interaction loci are restricted to lie within a certain distance of one or more atoms, residues, or other components of the target biopolymer molecular subset.

13. The method of claim 1, wherein estimating the location of one or more active sites further includes an analysis of additional information.

14. The method of claim 13, wherein the additional information includes homology data of the target biopolymer molecular subset to one or more other biopolymer molecular subsets with an already known or estimated active site.

15. The method of claim 13, wherein the target biopolymer includes a part or whole of a protein and the additional information involves knowledge of conserved regions or motifs involving the target protein or one or more related protein family members.

16. The method of claim 13, wherein the target biopolymer includes a part or whole of a protein and the additional information involves chemogenomics information for both the target protein and one or more related protein family members.

17. The method of claim 1, wherein sampling includes generating and exploring of configurations for each molecular combination via rigid-body transformations.

18. The method of claim 17, wherein computing the affinity scores includes the computation of shape complementarity of molecular surfaces of the configurations.

19. The method of claim 18, wherein an affinity score includes a shape complementarity score obtained via utilization of basis expansions for internal and external shape volume functions.

20. The method of claim 17, wherein computing of an affinity score includes the computation of electrostatic affinity of the two molecular subsets.

21. The method of claim 20, wherein an affinity score includes an electrostatic affinity score obtained via utilization of basis expansions for charge distributions and electrostatic potential fields.

22. The method of claim 17, wherein computing of an affinity score includes the joint computation of both shape complementarity and electrostatic affinity between two molecular subsets.

23. The method of claim 22, wherein the computation of shape complementarity or electrostatic affinity between two molecular subsets includes utilizing a basis expansion representing internal and external shape volume functions, charge density, and electrostatic potential functions associated with the two molecular subsets.

24. The method of claim 1, wherein analyzing the affinity scores includes subjecting a set of computed affinity scores to a set of decision criteria in order to select at least one optimal affinity score along with the corresponding molecular configurations for each molecular combination.

25. The method of claim 24, wherein the decision criteria are such that all molecular configurations for a molecular combination with affinity scores above a preset numerical threshold are selected.

26. The method of claim 24, wherein the decision criteria are such that molecular configurations for a molecular combination are sorted or rank prioritized based on their affinity scores, and further comprising selecting a fraction of the molecular configurations based on their sorted order.

27. The method of claim 24, wherein the decision criteria are such that molecular configurations for a molecular combination are selected based on a statistical analysis of the affinity scores.

28. The method of claim 27, wherein the decision criteria are based on an adaptive threshold dependent on observed statistics of the affinity scores for the configurations as they are sampled.

29. The method of claim 24, wherein the decision criteria are such that molecular configurations for a molecular combination are selected based on a cluster analysis of the molecular configurations involving both their affinity scores and their positional coordinates.

30. The method of claim 1, wherein determining one or more interaction loci includes examining the favorable configurations for each sampled molecular combination according to decision criteria in order to accept or reject molecular combinations based on relative comparisons of affinity scores.

31. The method of claim 30, wherein the affinity scores for different combinations featuring different molecular subsets from the collection, are normalized prior to application of the decision criteria.

32. The method of claim 31, wherein the normalization makes provisions for differences in size and/or degree of conformational flexibility of the molecular subsets when comparing across different molecular combinations.

33. The method of claim 30, wherein determining one or more interaction loci includes constructing a consensus based on the accepted molecular combinations.

34. The method of claim 33, wherein the consensus is used to assign a qualitative or quantitative interaction measure or score to each interaction locus.

35. The method of claim 33, wherein the consensus is built based on a spatial overlap of favorable molecular configurations from accepted molecular combinations.

36. The method of claim 35, wherein the affinity scores corresponding to the favorable molecular configurations from accepted molecular combinations are also included.

37. The method of claim 35, wherein the consensus is built based on the construction of a spatial occupancy grid that records a measure of overlap of one or more structural components of favorable configurations of accepted molecular combinations with each grid cell.

38. The method of claim 37, wherein the structural components are constituent atoms or bonds of the molecular subset.

39. The method of claim 37, wherein the measure of overlap is a frequency.

40. The method of claim 37, wherein the overlap measure is a weighted sum or average for each grid cell that is based on the affinity scores that correspond to overlapping favorable configurations of accepted molecular combinations.

41. The method of claim 37, wherein a smoothing operator or other suitable filter function is applied to the grid measure.

42. The method of claim 37, wherein the contribution of each favorable configuration of an accepted molecular combination to a given grid cell is further normalized based on the frequency of occurrence of the relevant overlapping portion or whole of the molecular subset in the collection of molecular subsets.

43. The method of claim 37, further comprising:
selecting grid cells based on a selection criteria; and
assigning interaction loci to accepted grid cells.

44. The method of claim 43, wherein the selection criteria is all grid cells with overlap measures above a preset threshold.

45. The method of claim 43, wherein the selection criteria includes a statistical analysis of the grid cells and their values.

46. The method of claim 43, wherein the interaction loci are assigned to the center points of the accepted grid cells.

47. The method of claim 43, further comprising constraining each interaction locus to be a certain minimum distance from all other interaction loci.

48. The method of claim 47, wherein assigning interaction loci based on the constructed grid is made via a greedy method for which each interaction locus is associated in succession to one or more of the best available high scoring grid cells.

49. The method of claim 1, wherein estimating the location of an active site is accomplished by determining a location near the molecular surface of the target biopolymer for which a sufficiently high intensity of interaction loci are found to occur within a volumetric region containing the location.

50. The method of claim 49, wherein a union of all such locations is used to determine one or more active sites.

51. The method of claim 49, wherein the intensity is a density or number of interaction loci.

52. The method of claim 49, wherein the intensity is a sum or other function of the intensity of interaction measures of interaction loci residing in the volumetric region.

53. The method of claim 49, wherein the volumetric region is a sphere of specified radius centered on the location.

54. The method of claim 49, wherein a radius and/or morphology of the volumetric region depends on the location and nature of the neighboring molecular surface of the target biopolymer molecular subset.

55. The method of claim 49, wherein a post-processing smoothing filter or other suitable function is applied to the passing locations prior to the final determination of active sites in order to reduce artificial discontinuities in the resultant volumetric regions.

56. The method of claim 1, wherein estimating a location of one or more active sites includes using a machine learning protocol to analyze the collection of interaction loci with their attendant interaction measures.

57. The method of claim 56, wherein the machine learning protocol is trained on one or more appropriate sets of biopolymers with known, well-characterized active sites.

58. The method of claim 56, wherein the machine learning protocol utilizes other additional descriptors related to sequence, structural, or functional motifs.

59. The method of claim 1, further comprising:
providing a computational system for estimating one or more actives sites of a target biopolymer molecular subset, wherein the system comprises one or more of a general purpose programmable computer including software to implement the computational platform, dedicated hardware, firmware, or a combination thereof.

60. An information storage medium having a plurality of instructions adapted to direct an information processing device to perform an operation estimating a location of one or more active sites on a target biopolymer molecular subset by analyzing collective results for a likelihood of molecular interaction between a collection of molecular subsets and the target biopolymer molecular subset, the operation comprising the steps of:
receiving input molecular data comprising at least one of structural, physical, or chemical information for the target biopolymer and the collection of molecular subsets;
defining an affinity function having a score that represents a likelihood of a molecular interaction between two molecular subsets for a given molecular configuration, wherein the affinity function includes the input molecular data;
sampling a set of molecular configurations for each of a plurality of molecular combinations, each combination involving the target biopolymer molecular subset and one of the molecular subsets from the collection;
computing a plurality of affinity scores, each associated with a sampled configuration;
analyzing the affinity scores, wherein analyzing the affinity scores includes determining one or more favorable molecular configurations associated with each molecular combination;
determining one or more interaction loci based on the favorable configurations for each sampled molecular combination, wherein an interaction locus is associated with at least one region of the target biopolymer molecular subset having a high likelihood of molecular interaction with one or more molecular subsets from the collection; and
estimating a location of one or more active sites on the target biopolymer molecular subset by analyzing the one or more interaction loci derived from the plurality of favorable configurations.

61. An apparatus for estimating the location of one or more active sites on a target biopolymer molecular subset by analyzing collective results for a likelihood of molecular interaction between a collection of molecular subsets and the target biopolymer molecular subset, the apparatus comprising:
an input for receiving input molecular data comprising at least one of structural, physical, or chemical information for the target biopolymer and the collection of molecular subsets;
an affinity function engine that computes a desired affinity function for a molecular configuration between two molecular subsets, wherein an affinity function includes an affinity score that represents a likelihood of molecular interaction between two molecular subsets for a molecular configuration;
a simulation engine that samples a set of molecular configurations for each of a plurality of molecular combinations, each combination involving the target biopolymer molecular subset and each of a plurality of molecular subsets from the collection, and that utilizes the affinity function engine to generate affinity scores for each molecular configuration sampled;
a molecular combination analysis engine that analyzes the set of affinity scores associated with the sampled molecular configurations for each molecular combination to determine one or more favorable molecular configurations with high affinity scores for each molecular combination; and
a library analysis engine that:
examines the favorable molecular configurations with their corresponding affinity function scores for each of the molecular combinations;
determines one or more interaction loci that represent regions of the target biopolymer molecular subset having a high likelihood of molecular interaction with one or more molecular subsets from the collection; and
estimates a location of one or more active sites on the target biopolymer molecular subset by analyzing the one or more interaction loci, wherein a subset of the union of the interaction loci are in, near, or cover portions of one or more active sites on the target biopolymer molecular subset.
